(19) United States
(12) Patent Application Publication
(10) Pub. No.: US 2026/0259170 A1
(43) Pub. Date: Sep. 3, 2026

(54) SENSOR ELEMENT AND GAS SENSOR

(71) Applicant: NGK INSULATORS, LTD., Nagoya-shi (JP)

(72) Inventors: Yusuke WATANABE, Nagoya-shi (JP); Masayuki UCHIDA, Nagoya-shi (JP); Yohei GORO, Nagoya-shi (JP)

(21) Appl. No.: 19/553,879

(22) Filed: Mar. 2, 2026

(30) Foreign Application Priority Data
Mar. 3, 2025 (JP) 2025-033071

(51) Int. Cl. G01N 27/406; G01N 27/41; G01N 33/00

(52) U.S. Cl. CPC G01N 27/4062; G01N 27/41; G01N 33/0027

(57) ABSTRACT

A sensor element includes an element body having formed therein a reference gas chamber in which a reference gas is to be stored; a first pump cell being configured to pump oxygen from around a first pump electrode to around a first reference electrode; a first inner lead portion configured to electrically connect a first connector electrode and the first reference electrode. The first inner lead portion has a reference gas flow region that is porous and constitutes at least part of a reference gas flow path allowing the reference gas to flow between the reference gas chamber and an outside of the element body. A limiting current A when oxygen is pumped out from around the first reference electrode to around the first pump electrode is 1.5 μA or more.

### CROSS-REFERENCE TO RELATED APPLICATION

The present application claims priority from Japanese Patent Application No. 2025-033071 filed Mar. 3, 2025, the entire contents of which are incorporated herein by reference.

### BACKGROUND OF THE INVENTION

### 1. Field of the Invention

The present invention relates to a sensor element and a gas sensor.

### 2. Description of the Related Art

Sensor elements are known in the related art for detecting a concentration of a specific gas such as a NOx concentration in a measurement gas such as an exhaust gas of an automobile (e.g., PTLs 1 and 2). PTL 1 describes a sensor element including an element body including solid electrolyte layers, a first measurement chamber disposed inside the element body, a detection electrode disposed in the first measurement chamber, a reference oxygen chamber disposed inside the element body, and a reference electrode disposed in the reference oxygen chamber. It is also described that a current is caused to flow between the detection electrode and the reference electrode to feed oxygen into the reference oxygen chamber. PTL 2 describes that a lead connected to a reference electrode has gas permeability to degas oxygen stored in the reference electrode. Accordingly, it is possible to prevent oxygen from being excessively stored in the reference electrode.

### CITATION LIST

### Patent Literature

PTL 1: JP 2018-100961 A
PTL 2: JP 2014-52363 A

### SUMMARY OF THE INVENTION

However, even in the case where the lead connected to the reference electrode has gas permeability as in PTL 2, there are cases in which the oxygen concentration around the reference electrode increases due to oxygen supplied to the vicinity of the reference electrode.

The present invention has been made to solve such a problem, and a main object of the present invention is to suppress an increase in oxygen concentration around the reference electrode due to the pumping of oxygen by a pump cell.

To achieve the main object described above, the present invention is configured as follows.

[1] A sensor element according to the present invention is a sensor element for detecting a concentration of a specific gas in a measurement gas, the sensor element including: an element body having an oxygen-ion-conductive solid electrolyte layer and having formed therein a measurement gas flow path and a reference gas chamber, the measurement gas flow path being a path into and through which the measurement gas is introduced and flows, the reference gas chamber being a chamber in which a reference gas used as a reference to detect the concentration of the specific gas in the measurement gas is to be stored; a first pump cell having a first pump electrode disposed at a portion of the element body that comes into contact with the measurement gas, and a first reference electrode disposed in the reference gas chamber, the first pump cell being configured to pump oxygen from around the first pump electrode to around the first reference electrode; a first connector electrode disposed on an outer surface of the element body; and a first inner lead portion disposed inside the element body, connected to the first reference electrode, and configured to electrically connect the first connector electrode and the first reference electrode; wherein the first inner lead portion has a reference gas flow region that is porous and constitutes at least part of a reference gas flow path allowing the reference gas to flow between the reference gas chamber and an outside of the element body; and wherein a limiting current A when oxygen is pumped out from around the first reference electrode to around the first pump electrode is 1.5 μA or more.

In the sensor element, the first inner lead portion has the reference gas flow region that is porous and constitutes at least part of the reference gas flow path allowing the reference gas to flow between the reference gas chamber and the outside of the element body. Further, the limiting current A when oxygen is pumped out from around the first reference electrode to around the first pump electrode is 1.5 μA or more. The limiting current A is positively correlated with the inverse of the diffusion resistance of the reference gas flow path; the larger the limiting current A, the smaller the diffusion resistance of the reference gas flow path. Because the limiting current A is 1.5 μA or more, the diffusion resistance between the outside of the element body and the reference gas chamber is not excessive, so that an increase in the oxygen concentration around the first reference electrode due to the pumping of oxygen by the first pump cell can be suppressed.

[2] In the sensor element described above (the sensor element according to [1]), the limiting current A may be 2.0 μA or more. In this way, the increase in the oxygen concentration around the first reference electrode due to the pumping of oxygen by the first pump cell can be further suppressed.

[3] In the sensor element described above (the sensor element according to [1] or [2]), the first inner lead portion may have a low-porosity region constituting part of the reference gas flow path, and a high-porosity region having a porosity higher than that of the low-porosity region and connecting the low-porosity region and the first reference electrode. In this way, the presence of the low-porosity region in part of the reference gas flow path can suppress some gas outside the sensor element from passing through the reference gas flow path and reaching the reference gas chamber, thereby suppressing a reduction in the accuracy of detection of the concentration of the specific gas. In addition, since the first inner lead portion has the high-porosity region between the low-porosity region and the first reference electrode, the high-porosity region functions as a buffer that suppresses abrupt changes in the oxygen concentration in the reference gas chamber, thereby stabilizing the oxygen concentration in the reference gas chamber.

[4] In the sensor element described above (the sensor element according to [3]), a void volume Vh inside the high-porosity region may be 0.5×10⁻³ mm³ or more. In this way, the high-porosity region can more reliably exhibit the buffer function described above.

[5] In the sensor element described above (the sensor element according to [4]), the void volume Vh may be 1.0×10⁻³ mm³ or more. In this way, the high-porosity region provides a higher effect of stabilizing the oxygen concentration in the reference gas chamber.

[6] The sensor element described above (the sensor element according to any one of [1] to [5]) may further include: a second pump cell having a second pump electrode disposed at a portion of the element body that comes into contact with the measurement gas, and a second reference electrode disposed in the reference gas chamber, the second pump cell being configured to pump oxygen from around the second pump electrode to around the second reference electrode; a second connector electrode disposed on an outer surface of the element body; and a second inner lead portion disposed inside the element body, connected to the second reference electrode, and configured to electrically connect the second connector electrode and the second reference electrode.

[7] A gas sensor according to the present invention is a gas sensor including the sensor element described above (the sensor element according to any one of [1] to [6]). The gas sensor provides the same effects as the sensor element described above, e.g., suppressing an increase in the oxygen concentration around the first reference electrode due to the pumping of oxygen by the first pump cell. The gas sensor may further include a case in which the sensor element is disposed, and a sealing member that seals an end portion of the case.

### BRIEF DESCRIPTION OF THE DRAWINGS

FIG. 1 is a longitudinal cross-sectional view of a gas sensor 100.

FIG. 2 is a schematic cross-sectional view schematically illustrating an example configuration of a sensor element 101.

FIG. 3 is a block diagram illustrating an electrical connection relationship between a control device 90 and each cell.

FIG. 4 is a perspective view of the vicinity of a rear end of an element body 102.

FIG. 5 is a partial cross-sectional view showing the vicinity of first and second inner lead portions 61, 65 inside the sensor element 101.

FIG. 6 is an enlarged cross-sectional view showing the vicinity of a reference gas chamber 49.

FIG. 7 is a partial cross-sectional view taken along line A-A of FIG. 5.

FIG. 8 is a partial cross-sectional view of a sensor element 201 according to a modification.

FIG. 9 is a schematic cross-sectional view of a sensor element 301 according to a modification.

### DETAILED DESCRIPTION OF THE INVENTION

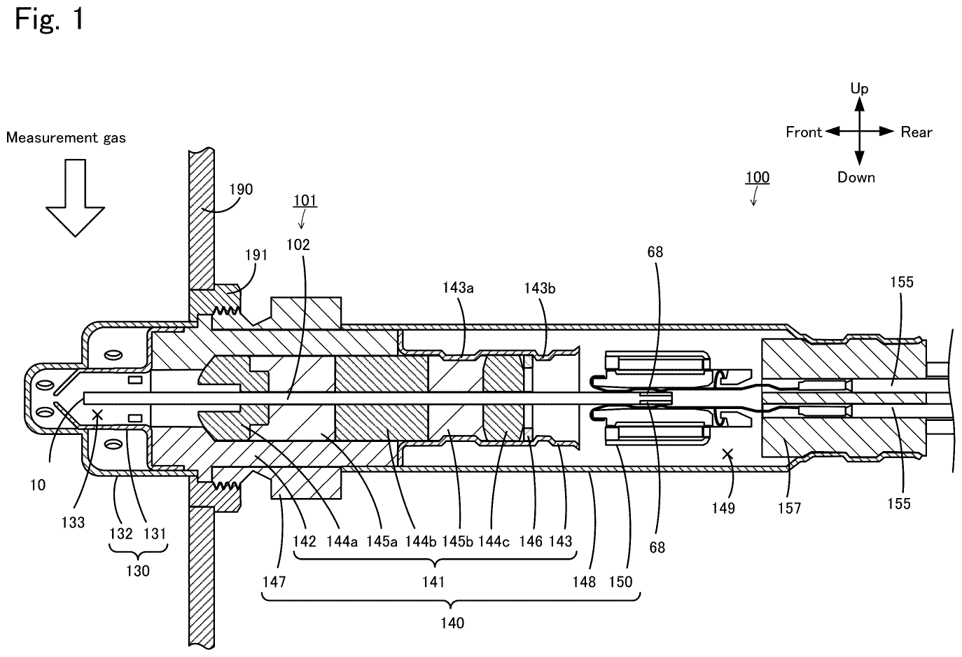

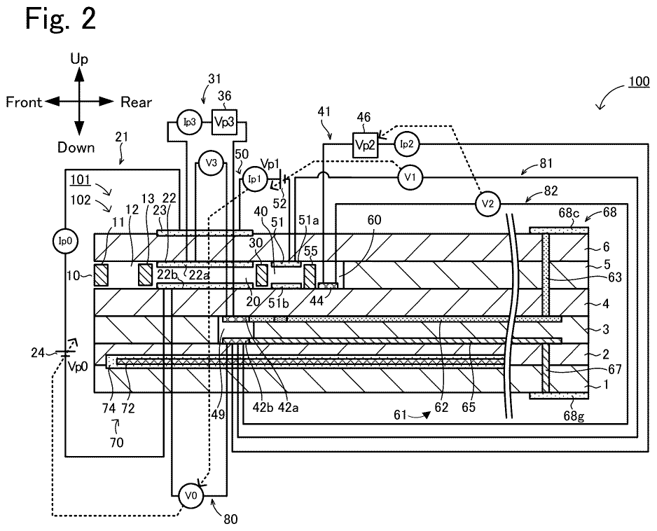

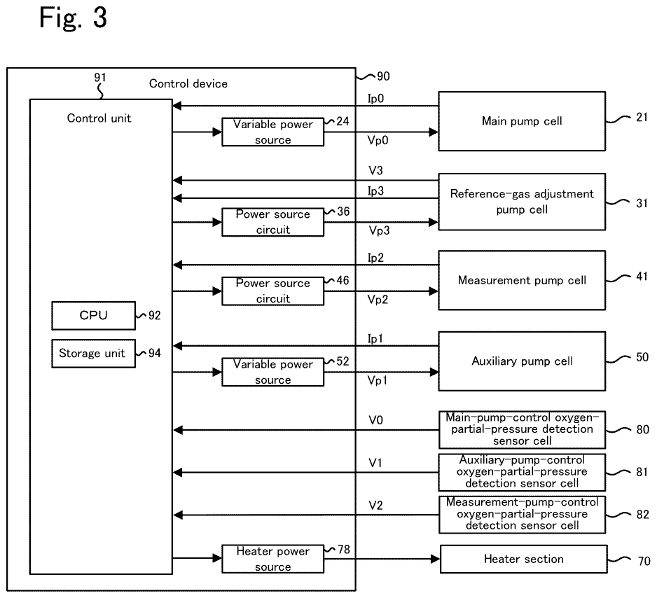

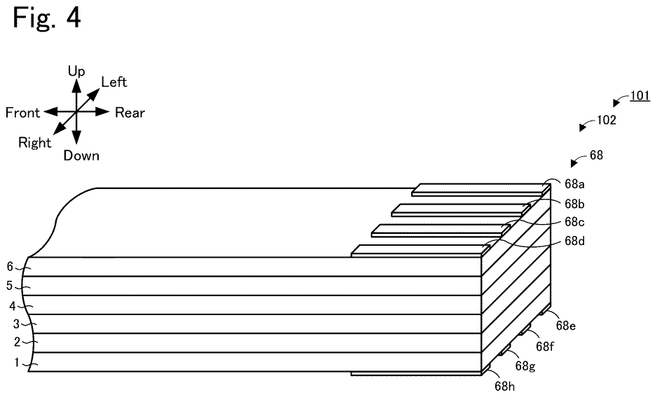

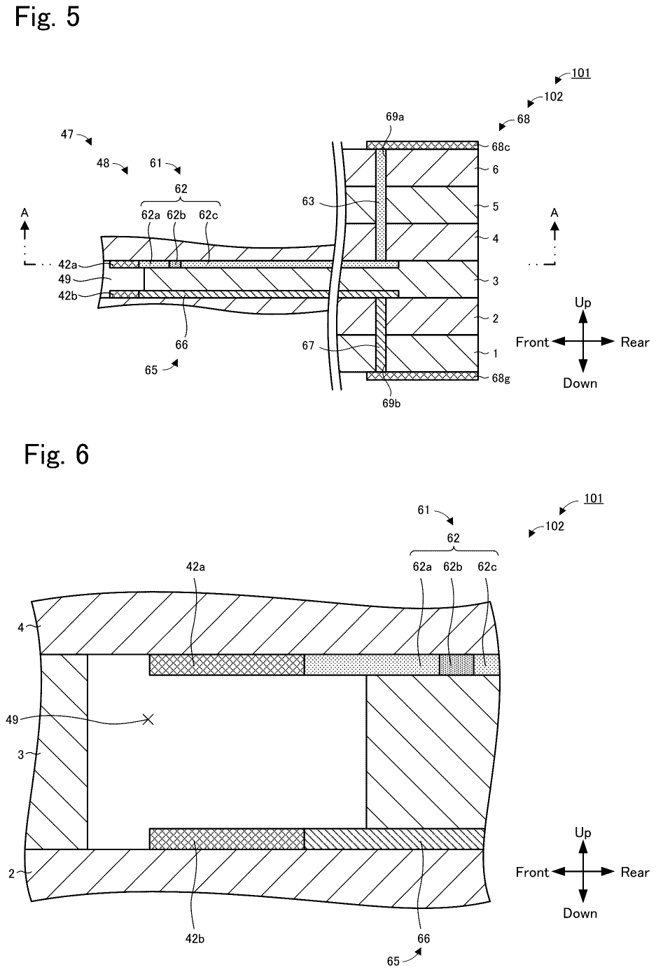

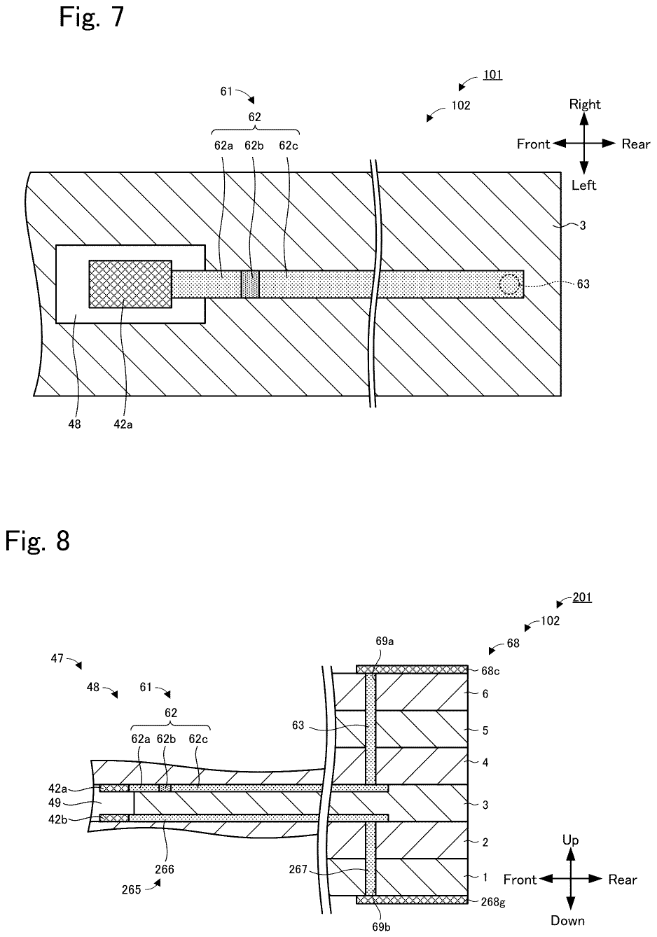

An embodiment of the present invention will now be described with reference to the drawings. FIG. 1 is a longitudinal sectional view of a gas sensor 100 according to an embodiment of the present invention. FIG. 2 is a schematic sectional view schematically illustrating an example configuration of a sensor element 101 included in the gas sensor 100. FIG. 3 is a block diagram illustrating an electrical connection relationship between a control device 90 and each cell. FIG. 4 is a perspective view of the vicinity of a rear end of an element body 102. FIG. 5 is a partial cross-sectional view showing the vicinity of first and second inner lead portions 61, 65 inside the sensor element 101. FIG. 6 is an enlarged cross-sectional view showing the vicinity of a reference gas chamber 49. FIG. 7 is a partial cross-sectional view taken along line A-A of FIG. 5. The sensor element 101 has an element body 102 that has an elongated rectangular parallelepiped shape. The longitudinal direction of the element body 102 (the left-right direction in FIG. 2) is represented as a front-rear direction, and the thickness direction of the element body 102 (the up-down direction in FIG. 2) is represented as an up-down direction. The width direction of the element body 102 (a direction perpendicular to the front-rear direction and the up-down direction) is represented as a left-right direction.

As illustrated in FIG. 1, the gas sensor 100 includes the sensor element 101 that includes the element body 102, a protective cover 130 that protects the front end side of the element body 102, and a sensor assembly 140. The sensor assembly 140 includes a connector 150 to be brought into electrical conduction with the sensor element 101. As illustrated in FIG. 1, the gas sensor 100 is attached to, for example, a pipe 190 such as an exhaust gas pipe of a vehicle and is used to measure the concentration of a specific gas such as NOx or Oz contained in an exhaust gas serving as a measurement gas. In this embodiment, the gas sensor 100 is configured to measure a NOx concentration as a concentration of a specific gas.

The protective cover 130 includes a bottomed cylindrical inner protective cover 131 that covers a front end of the element body 102, and a bottomed cylindrical outer protective cover 132 that covers the inner protective cover 131. The inner protective cover 131 and the outer protective cover 132 have formed therein a plurality of holes through which the measurement gas flows into the protective cover 130. A sensor element chamber 133 is formed as a space surrounded by the inner protective cover 131, and the front end of the element body 102 is arranged in the sensor element chamber 133.

The sensor assembly 140 includes an element sealing unit 141 that seals the sensor element 101 in a fixed manner, a bolt 147 attached to the element sealing unit 141, an outer cylinder 148, and the connector 150. The connector 150 is in contact with multiple connector electrodes 68 formed on surfaces (upper and lower surfaces) of a rear end of the element body 102 of the sensor element 101 and is electrically connected to the connector electrodes.

The element sealing unit 141 includes a cylindrical main metal fitting 142, a cylindrical inner cylinder 143 coaxially welded to the main metal fitting 142 in a fixed manner, and ceramic supporters 144a to 144c, compacts 145a and 145b, and a metal ring 146, which are sealed in a through hole inside the main metal fitting 142 and the inner cylinder 143. The sensor element 101 is located along the center axis of the element sealing unit 141 in such a manner as to extend through the element sealing unit 141 in the front-rear direction. The inner cylinder 143 has a diameter reduction section 143a for pressing the compact 145b in a direction toward the center axis of the inner cylinder 143, and a diameter reduction section 143b for pressing the ceramic supporters 144a to 144c and the compacts 145a and 145b to the front via the metal ring 146. The pressing forces from the diameter reduction sections 143a and 143b compress the compacts 145a and 145b between the sensor element 101 and the set of the main metal fitting 142 and the inner cylinder 143. Accordingly, the compacts 145a and 145b perform sealing between the sensor element chamber 133 in the protective cover 130 and a space 149 in the outer cylinder 148, and fix the sensor element 101.

The bolt 147 is coaxially fixed to the main metal fitting 142, and has a male threaded portion formed on an outer peripheral surface thereof. The male threaded portion of the bolt 147 is inserted into a fixing member 191 having a female threaded portion on an inner peripheral surface thereof. The fixing member 191 is welded to the pipe 190. Accordingly, the gas sensor 100 is fixed to the pipe 190 in such a manner that a portion of the gas sensor 100 corresponding to the front end of the element body 102 of the sensor element 101 and the protective cover 130 projects into the pipe 190.

The outer cylinder 148 surrounds the inner cylinder 143, the sensor element 101, and the connector 150. A plurality of lead wires 155 connected to the connector 150 are drawn out from a rear end of the outer cylinder 148. The lead wires 155 are in electrical conduction with electrodes (described below) of the sensor element 101 via the connector 150. A gap between the outer cylinder 148 and the lead wires 155 is sealed with a rubber stopper 157. The space 149 in the outer cylinder 148 is filled with a reference gas (in this embodiment, air). The rear end of the element body 102 of the sensor element 101 is arranged in the space 149.

As shown in FIG. 2, the sensor element 101 has the element body 102, cells 21, 31, 41, 50, and 80 to 82, and a heater section 70. The element body 102 is an element including a layered body having six layers, each of which is formed of an oxygen-ion-conductive solid electrolyte layer such as a zirconia (ZrO₂) layer. The six layers include a first substrate layer 1, a second substrate layer 2, a third substrate layer 3, a first solid electrolyte layer 4, a spacer layer 5, and a second solid electrolyte layer 6 and are stacked in the stated order from bottom to top in FIG. 2. The solid electrolyte forming the six layers is dense and gas-tight. The element body 102 is manufactured by, for example, after performing predetermined processing and circuit pattern printing on ceramic green sheets, each corresponding to one of the layers, stacking the ceramic green sheets, firing the stacked ceramic green sheets, and combining the fired ceramic green sheets together to form a single unit.

At the front end side of the element body 102, a gas inlet 10, a first diffusion rate-limiting section 11, a buffer space 12, a second diffusion rate-limiting section 13, a first internal cavity 20, a third diffusion rate-limiting section 30, a second internal cavity 40, a fourth diffusion rate-limiting section 55, and a third internal cavity 60 are formed adjacent and communicate in the stated order between a lower surface of the second solid electrolyte layer 6 and an upper surface of the first solid electrolyte layer 4.

The gas inlet 10, the buffer space 12, the first internal cavity 20, the second internal cavity 40, and the third internal cavity 60 are internal spaces of the sensor element 101, which are formed by removing a portion of the spacer layer 5, with the tops thereof defined by the lower surface of the second solid electrolyte layer 6, the bottoms thereof defined by the upper surface of the first solid electrolyte layer 4, and the sides thereof defined by the side surfaces of the spacer layer 5.

The first diffusion rate-limiting section 11, the second diffusion rate-limiting section 13, and the third diffusion rate-limiting section 30 are each provided as two horizontally long slits (whose openings have a longitudinal direction along a direction perpendicular to the plane of FIG. 2). The fourth diffusion rate-limiting section 55 is disposed as a single horizontally long slit (whose opening has a longitudinal direction along a direction perpendicular to the plane of FIG. 2), which is formed as a gap from the lower surface of the second solid electrolyte layer 6. Note that the portion from the gas inlet 10 up to the third internal cavity 60 is also referred to as a measurement gas flow section.

A reference gas chamber 49 is disposed between a lower surface of the first solid electrolyte layer 4 and an upper surface of the second substrate layer 2. The reference gas chamber 49 is an internal space of the element body 102, which is formed by removing a portion of the third substrate layer 3. The reference gas chamber 49 is formed as an independent space that is not open to outside the element body 102, unlike the measurement gas flow section. The reference gas chamber 49 is arranged underneath the first internal cavity 20. The reference gas chamber 49 is configured to store a reference gas serving as a reference to measure the NOx concentration. The reference gas is a gas having a predetermined oxygen concentration and is atmospheric air in this embodiment. The reference gas chamber 49 is provided with a first reference electrode 42a and a second reference electrode 42b.

The first reference electrode 42a is a porous electrode disposed on the lower surface of the first solid electrolyte layer 4. The second reference electrode 42b is a porous electrode disposed on the upper surface of the second substrate layer 2. As described below, the second reference electrode 42b can be used to measure the oxygen concentration (oxygen partial pressure) in the first internal cavity 20, the second internal cavity 40, and the third internal cavity 60. The first reference electrode 42a and the second reference electrode 42b are each formed as a porous cermet electrode (e.g., a cermet electrode composed of Pt and Zro₂).

In the measurement gas flow section, the gas inlet 10 is a portion open to an external space such that the measurement gas is taken into the sensor element 101 from the external space through the gas inlet 10. The first diffusion rate-limiting section 11 is a portion that applies a predetermined diffusion resistance to the measurement gas taken through the gas inlet 10. The buffer space 12 is a space provided to guide the measurement gas introduced through the first diffusion rate-limiting section 11 to the second diffusion rate-limiting section 13. The second diffusion rate-limiting section 13 is a portion that applies a predetermined diffusion resistance to the measurement gas to be introduced into the first internal cavity 20 from the buffer space 12. When the measurement gas is introduced into the first internal cavity 20 from outside the sensor element 101, the measurement gas, which is rapidly taken into the sensor element 101 through the gas inlet 10 due to changes in the pressure of the measurement gas in the external space (pulsations in exhaust pressure in a case where the measurement gas is an exhaust gas of an automobile), is not directly introduced into the first internal cavity 20, but is introduced into the first internal cavity 20 after the changes in the pressure of the measurement gas are compensated for through the first diffusion rate-limiting section 11, the buffer space 12, and the second diffusion rate-limiting section 13. Consequently, the changes in the pressure of the measurement gas to be introduced into the first internal cavity 20 are almost negligible. The first internal cavity 20 is provided as a space for adjusting the oxygen partial pressure in the measurement gas introduced through the second diffusion rate-limiting section 13. The oxygen partial pressure is adjusted by the operation of a main pump cell 21.

The main pump cell 21 is an electrochemical pump cell including an inner pump electrode 22 having a ceiling electrode portion 22a disposed over substantially the entire lower surface of a portion of the second solid electrolyte layer 6 facing the first internal cavity 20, an outer pump electrode 23 disposed in a region on an upper surface of the second solid electrolyte layer 6 corresponding to the ceiling electrode portion 22a in such a manner that the outer pump electrode 23 is exposed to the outside of the element body 102 (the sensor element chamber 133 in FIG. 1), and the second solid electrolyte layer 6 held between the electrodes 22 and 23.

The inner pump electrode 22 is formed across the upper and lower solid electrolyte layers defining the first internal cavity 20 (i.e., the second solid electrolyte layer 6 and the first solid electrolyte layer 4), and the spacer layer 5 forming the sidewalls. Specifically, the ceiling electrode portion 22a is formed on the lower surface of the second solid electrolyte layer 6, which forms a ceiling surface of the first internal cavity 20. A bottom electrode portion 22b is formed directly on the upper surface of the first solid electrolyte layer 4, which forms a bottom surface of the first internal cavity 20. Side electrode portions (not illustrated) are formed on sidewall surfaces (inner surfaces) of the spacer layer 5, which form both sidewall portions of the first internal cavity 20, so as to connect the ceiling electrode portion 22a and the bottom electrode portion 22b to each other. The inner pump electrode 22 is thus disposed to have a tunnel structure in the portion where the side electrode portions are disposed.

The inner pump electrode 22 and the outer pump electrode 23 are each formed as a porous cermet electrode (e.g., a cermet electrode composed of Pt and ZrO₂ containing 1% Au). The inner pump electrode 22, which comes into contact with the measurement gas, is formed of a material having lowered reduction ability for the NOx component in the measurement gas.

In the main pump cell 21, a desired pump voltage Vp0 is applied between the inner pump electrode 22 and the outer pump electrode 23 to cause a pump current Ip0 to flow between the inner pump electrode 22 and the outer pump electrode 23 in the positive direction or the negative direction. Accordingly, the main pump cell 21 is capable of pumping out oxygen to the external space from the first internal cavity 20 or pumping oxygen into the first internal cavity 20 from the external space.

Further, the inner pump electrode 22, the second solid electrolyte layer 6, the spacer layer 5, the first solid electrolyte layer 4, the third substrate layer 3, the second substrate layer 2, and the second reference electrode 42b form an electrochemical sensor cell, namely, a main-pump-control oxygen-partial-pressure detection sensor cell 80, for detecting the oxygen concentration (oxygen partial pressure) in the atmosphere in the first internal cavity 20.

An electromotive force (voltage V0) in the main-pump-control oxygen-partial-pressure detection sensor cell 80 is measured to determine the oxygen concentration (oxygen partial pressure) in the first internal cavity 20. In addition, feedback control is performed on the pump voltage Vp0 of a variable power source 24 so that the electromotive force V0 becomes a target value to control the pump current Ip0. Accordingly, the oxygen concentration in the first internal cavity 20 can be kept at a predetermined constant value.

The third diffusion rate-limiting section 30 is a portion that applies a predetermined diffusion resistance to the measurement gas in which the oxygen concentration (oxygen partial pressure) is controlled in the first internal cavity 20 by the operation of the main pump cell 21 to guide the measurement gas into the second internal cavity 40.

The second internal cavity 40 is provided as a space for, after the adjustment of the oxygen concentration (oxygen partial pressure) in the first internal cavity 20 in advance, further adjusting the oxygen partial pressure in the measurement gas introduced through the third diffusion rate-limiting section 30 by using an auxiliary pump cell 50. Accordingly, the oxygen concentration in the second internal cavity 40 can be kept constant with high accuracy, and thus the gas sensor 100 can accurately measure the NOx concentration.

The auxiliary pump cell 50 is an auxiliary electrochemical pump cell including an auxiliary pump electrode 51 having a ceiling electrode portion 51a disposed over substantially the entire lower surface of a portion of the second solid electrolyte layer 6 facing the second internal cavity 40, the outer pump electrode 23 (not limited to the outer pump electrode 23; any suitable electrode disposed on the outer peripheral surface of the element body 102 suffices), and the second solid electrolyte layer 6.

The auxiliary pump electrode 51 has a tunnel structure similar to that of the inner pump electrode 22 disposed in the first internal cavity 20 described above, and is disposed in the second internal cavity 40. That is, the ceiling electrode portion 51a is formed on the lower surface of the second solid electrolyte layer 6, which forms a ceiling surface of the second internal cavity 40. A bottom electrode portion 51b is formed directly on the upper surface of the first solid electrolyte layer 4, which forms a bottom surface of the second internal cavity 40. Side electrode portions (not illustrated) connecting the ceiling electrode portion 51a and the bottom electrode portion 51b to each other are formed on both sidewall surfaces of the spacer layer 5, which form sidewalls of the second internal cavity 40. Thus, the tunnel structure is provided. Like the inner pump electrode 22, the auxiliary pump electrode 51 is also formed of a material having lowered reduction ability for the NOx component in the measurement gas.

In the auxiliary pump cell 50, a desired voltage Vp1 is applied between the auxiliary pump electrode 51 and the outer pump electrode 23. Accordingly, the auxiliary pump cell 50 is capable of pumping out oxygen in the atmosphere in the second internal cavity 40 to the external space or pumping oxygen into the second internal cavity 40 from the external space.

Further, the auxiliary pump electrode 51, the second reference electrode 42b, the second solid electrolyte layer 6, the spacer layer 5, the first solid electrolyte layer 4, the third substrate layer 3, and the second substrate layer 2 form an electrochemical sensor cell, namely, an auxiliary-pump-control oxygen-partial-pressure detection sensor cell 81, for controlling the oxygen partial pressure in the atmosphere in the second internal cavity 40.

The auxiliary pump cell 50 performs pumping using a variable power source 52 whose voltage is controlled on the basis of an electromotive force (voltage V1) detected by the auxiliary-pump-control oxygen-partial-pressure detection sensor cell 81. Accordingly, the oxygen partial pressure in the atmosphere in the second internal cavity 40 is controlled to a low partial pressure that does not substantially affect NOx measurement.

Additionally, a pump current Ip1 is used to control the electromotive force of the main-pump-control oxygen-partial-pressure detection sensor cell 80. Specifically, the pump current Ip1 is input as a control signal to the main-pump-control oxygen-partial-pressure detection sensor cell 80, for which the target value of the voltage V0 described above is controlled to perform control so that the gradient of the oxygen partial pressure in the measurement gas to be introduced into the second internal cavity 40 from the third diffusion rate-limiting section 30 remains always constant. When the gas sensor 100 is used as a NOx sensor, the oxygen concentration in the second internal cavity 40 is kept at a constant value of about 0.001 ppm by the operation of the main pump cell 21 and the auxiliary pump cell 50.

The fourth diffusion rate-limiting section 55 is a portion that applies a predetermined diffusion resistance to the measurement gas in which the oxygen concentration (oxygen partial pressure) is controlled in the second internal cavity 40 by the operation of the auxiliary pump cell 50 to guide the measurement gas into the third internal cavity 60. The fourth diffusion rate-limiting section 55 serves to limit the amount of NOx flowing into the third internal cavity 60.

The third internal cavity 60 is provided as a space for, after the adjustment of the oxygen concentration (oxygen partial pressure) in the second internal cavity 40 in advance, performing a process on the measurement gas introduced through the fourth diffusion rate-limiting section 55 to measure the nitrogen oxide (NOx) concentration in the measurement gas. The measurement of the NOx concentration is mainly performed in the third internal cavity 60 by the operation of a measurement pump cell 41.

The measurement pump cell 41 measures the NOx concentration in the measurement gas in the third internal cavity 60. The measurement pump cell 41 is an electrochemical pump cell including a measurement electrode 44 disposed directly on a portion of the upper surface of the first solid electrolyte layer 4 facing the third internal cavity 60, the second reference electrode 42b, the first solid electrolyte layer 4, the third substrate layer 3, and the second substrate layer 2. The measurement electrode 44 is a porous cermet electrode (e.g., a cermet electrode composed of Pt and ZrO₂) composed of a material having higher reduction ability for the NOx component in the measurement gas than the material of the inner pump electrode 22. The measurement electrode 44 also functions as a NOx reducing catalyst for reducing NOx present in the atmosphere in the third internal cavity 60.

The measurement pump cell 41 is capable of pumping oxygen produced by decomposition of nitrogen oxide in the atmosphere around the measurement electrode 44 into around the second reference electrode 42b, that is, into the reference gas chamber 49, and detecting the amount of produced oxygen as a pump current Ip2.

Further, the measurement electrode 44, the second reference electrode 42b, the first solid electrolyte layer 4, the third substrate layer 3, and the second substrate layer 2 form an electrochemical sensor cell, namely, a measurement-pump-control oxygen-partial-pressure detection sensor cell 82, for detecting the oxygen partial pressure around the measurement electrode 44. A power source circuit 46 is controlled on the basis of an electromotive force (voltage V2) detected by the measurement-pump-control oxygen-partial-pressure detection sensor cell 82.

The measurement gas guided into the second internal cavity 40, in which the oxygen partial pressure is controlled, passes through the fourth diffusion rate-limiting section 55 and reaches the measurement electrode 44 in the third internal cavity 60. In the measurement gas around the measurement electrode 44, nitrogen oxide is reduced to produce oxygen (2NO→N₂+O₂). The produced oxygen is pumped by the measurement pump cell 41. In the pumping process, a voltage Vp2 of the power source circuit 46 is controlled so that the voltage V2 detected by the measurement-pump-control oxygen-partial-pressure detection sensor cell 82 becomes constant (a target value). Since the amount of oxygen produced around the measurement electrode 44 is proportional to the nitrogen oxide concentration in the measurement gas, the nitrogen oxide concentration in the measurement gas is calculated using the pump current Ip2 in the measurement pump cell 41.

Further, the second solid electrolyte layer 6, the spacer layer 5, the first solid electrolyte layer 4, the inner pump electrode 22, and the first reference electrode 42a form an electrochemical reference-gas adjustment pump cell 31. The reference-gas adjustment pump cell 31 performs pumping in response to the flow of a pump current Ip3 by the application of a voltage Vp3 from a power source circuit 36 connected between the inner pump electrode 22 and the first reference electrode 42a. Accordingly, the reference-gas adjustment pump cell 31 pumps oxygen into the space around the first reference electrode 42a, that is, the reference gas chamber 49, from the space around the inner pump electrode 22, that is, the first internal cavity 20. A voltage V3 of the reference-gas adjustment pump cell 31 can be measured.

In the gas sensor 100 having the configuration described above, the main pump cell 21 and the auxiliary pump cell 50 are activated to provide the measurement pump cell 41 with the measurement gas in which the oxygen partial pressure is always kept at a constant low value (a value that does not substantially affect NOx measurement). Accordingly, the NOx concentration in the measurement gas can be determined on the basis of the pump current Ip2 caused to flow by the measurement pump cell 41 pumping out oxygen produced by reducing NOx, approximately in proportion to the concentration of NOx in the measurement gas.

The heater portion 70 serves the role of temperature adjustment by heating the sensor element 101 and keeping a temperature of the sensor element 101 in order to enhance the oxygen-ion conductivity of the solid electrolyte. The heater portion 70 includes a heater 72 and a heater insulating layer 74.

The heater 72 is an electric resistor formed in a state sandwiched above and below by the first substrate layer 1 and the second substrate layer 2. When power is supplied from a heater power source 78, the heater 72 generates heat and heats the solid electrolyte of the element body 102 and keeps the temperature.

The heater 72 is embedded across an entire area from the first internal cavity 20 to the third internal cavity 60 and is capable of adjusting the temperature of the entire sensor element 101 to a temperature at which the solid electrolyte is activated.

The heater insulating layer 74 is an insulating layer composed of porous alumina, which is formed of an insulating material such as alumina on upper and lower surfaces of the heater 72. The heater insulating layer 74 is formed to provide electrical insulation between the first substrate layer 1 and the heater 72 and electrical insulation between the second substrate layer 2 and the heater 72.

As illustrated in FIG. 3, the control device 90 includes the variable power supplies 24 and 52 described above, the power source circuits 36 and 46 described above, the heater power source 78 described above, and a control unit 91. The control unit 91 is a microprocessor including a CPU 92 and a storage unit 94. The storage unit 94 is a rewritable nonvolatile memory capable of storing various programs and various data, for example. The control unit 91 receives the voltage V0 of the main-pump-control oxygen-partial-pressure detection sensor cell 80, the voltage V1 of the auxiliary-pump-control oxygen-partial-pressure detection sensor cell 81, the voltage V2 of the measurement-pump-control oxygen-partial-pressure detection sensor cell 82, the voltage V3 of the reference-gas adjustment pump cell 31, the pump current Ip0 flowing through the main pump cell 21, the pump current Ip1 flowing through the auxiliary pump cell 50, the pump current Ip2 flowing through the measurement pump cell 41, and the pump current Ip3 flowing through the reference-gas adjustment pump cell 31. The control unit 91 outputs control signals to the variable power supplies 24 and 52 to control the voltages Vp0 and Vp1, thereby controlling the main pump cell 21 and the auxiliary pump cell 50. The control unit 91 outputs control signals to the power source circuits 36 and 46 to control the voltages Vp3 and Vp2, thereby controlling the reference-gas adjustment pump cell 31 and the measurement pump cell 41. The control unit 91 outputs a control signal to the heater power source 78 to control the power to be supplied to the heater 72, thereby controlling the heater 72.

The control unit 91 performs feedback control of the pump voltage Vp0 of the variable power source 24 so that the voltage V0 becomes a target value V0* (i.e., the oxygen concentration in the first internal cavity 20 becomes a target concentration).

The control unit 91 performs feedback control of the voltage Vp1 of the variable power source 52 so that the voltage V1 becomes a target value V1* (i.e., the oxygen concentration in the second internal cavity 40 becomes a predetermined low oxygen concentration that does not substantially affect NOx measurement). Additionally, the control unit 91 sets (performs feedback control of) the target value V0* of the voltage V0 on the basis of the pump current Ip1 so that the pump current Ip1 flowing by the application of the voltage Vp1 becomes a target value Ip1*. Accordingly, the gradient of the oxygen partial pressure in the measurement gas to be introduced into the second internal cavity 40 from the third diffusion rate-limiting section 30 remains always constant.

The control unit 91 performs feedback control of the voltage Vp2 of the power source circuit 46 so that the voltage V2 becomes a target value V2* (i.e., the concentration of oxygen produced by reducing the nitrogen oxide in the measurement gas in the third internal cavity 60 becomes substantially zero), and calculates the NOX concentration in the measurement gas on the basis of the pump current Ip2. The target value V2* is determined in advance as a value at which the pump current Ip2 flowing by the application of the voltage Vp2 subjected to feedback control becomes a limiting current. As can be seen from FIG. 2, both the voltage Vp2 and the voltage V2 are basically voltages between the measurement electrode 44 and the second reference electrode 42b. During the application the voltage Vp2, the voltage V2 is affected by the voltage Vp2. Accordingly, preferably, the voltage Vp2 output from the power source circuit 46 is set to, for example, a voltage that is repeatedly turned on and off, such as a pulsed voltage, and the control unit 91 measures the value of the voltage V2 during a period in which the voltage Vp2 is off, and performs feedback control so that the voltage V2 becomes the target value V2*. When the pump current Ip2 flows, as described above, oxygen in the third internal cavity 60 is pumped into the reference gas chamber 49.

The control unit 91 controls the power source circuit 36 so that the constant voltage Vp3 (e.g., a DC voltage) is applied to the reference-gas adjustment pump cell 31, and causes the pump current Ip3 to flow. Accordingly, oxygen is pumped into the reference gas chamber 49 from the first internal cavity 20. The pump current Ip3 may be, for example, 1 μA or more and 10 μA or less. The voltage Vp3 may be a pulse voltage or another voltage that is repeatedly turned on and off. In this case, the pump current Ip3 becomes a periodic current such as a pulse current. When the pump current Ip3 is periodic, it suffices that its average value be 1 μA or more and 10 μA or less. Note that the control unit 91 can also control the reference-gas adjustment pump cell 31 by controlling the power source circuit 36 so as to pump oxygen out from the reference gas chamber 49 to the first internal cavity 20, i.e., to pump oxygen from around the first reference electrode 42a to the inside pump electrode 22.

Further, the control unit 91 measures the voltage V3 of the reference-gas adjustment pump cell 31, and measures the resistance value of the reference-gas adjustment pump cell 31 on the basis of the voltage V3 and the pump current Ip3. The control unit 91 performs heater control processing that controls the power supplied to the heater 72 by controlling the heater power source 78 based on the measured resistance value. For example, the control unit 91 calculates the temperature of the first solid electrolyte layer 4, which is a portion of the reference-gas adjustment pump cell 31, on the basis of the measured resistance value, and controls the heater power source 78 so that the temperature becomes a target temperature. Accordingly, the control unit 91 adjusts the temperature of the cells 21, 31, 41, 50, 80, 81, and 82 of the sensor element 101 (in particular, the temperature of the solid electrolyte layers forming portions of these cells) to a predetermined temperature at which the solid electrolyte is activated. The target temperature may be, for example, greater than or equal to 700° C. and less than or equal to 900° C.

The control unit 91 may measure the resistance value of the reference-gas adjustment pump cell 31 in a state where hardly any oxygen is being pumped into the reference gas chamber 49 by the reference-gas adjustment pump cell 31. In this case, the control unit 91 may control the power source circuit 36 to apply a relatively small voltage Vp3, and measure the resistance value of the reference-gas adjustment pump cell 31 on the basis of the weak values of the pump current Ip3 and the voltage V3 obtained at this time.

Including the variable power supplies 24 and 52, the power source circuits 36 and 46, and the heater power source 78 shown in FIG. 3, the control device 90 is electrically connected to the respective electrodes 22, 23, 42a, 42b, 44, and 51 and to the heater 72 via a plurality of connector electrodes 68.

The plurality of connector electrodes 68 each function as terminals for electrically conducting between the sensor element 101 and the outside. As shown in FIG. 4, the plurality of connector electrodes 68 are disposed on the rear-end side of the upper surface or lower surface of the element body 102 of the sensor element 101. Specifically, the plurality of connector electrodes 68 are connector electrodes 68a to 68d that are disposed in order from the left on the rear-end side of the upper surface of the element body 102, and connector electrodes 68e to 68h that are disposed in order from the left on the rear-end side of the lower surface of the element body 102. The connector electrodes 68a to 68d, 68g, and 68h are each electrically conducted to the measurement electrode 44, the outside pump electrode 23, the first reference electrode 42a, the auxiliary pump electrode 51, the second reference electrode 42b, and the auxiliary pump electrode 51 via lead portions disposed inside and/or on the outer surface of the element body 102. The connector electrodes 68e and 68f are each electrically conducted to the heater 72 via lead portions disposed inside and/or on the outer surface of the element body 102.

Hereinafter, among the lead portions that respectively connect the electrodes 22, 23, 42a, 42b, 44, and 51 and the heater 72 to the plurality of connector electrodes 68, a lead portion that connects the first reference electrode 42a and the connector electrode 68c, and a lead portion that connects the second reference electrode 42b and the connector electrode 68g, will be described in detail.

As shown in FIG. 5, the first reference electrode 42a and the connector electrode 68c (an example of a first connector electrode) are electrically conducted via a first inside lead portion 61 that is a lead portion disposed inside the element body 102. The second reference electrode 42b and the connector electrode 68g (an example of a second connector electrode) are electrically conducted via a second inside lead portion 65 that is a lead portion disposed inside the element body 102.

The first inside lead portion 61 is a conductor connected to the first reference electrode 42a, and electrically conducts between the connector electrode 68c and the first reference electrode 42a. The first inside lead portion 61 includes a first lead wire 62 and a first through-hole conductor 63. The first lead wire 62 is formed on the lower surface of the first solid electrolyte layer 4 and is disposed so as to extend in the front-rear direction between the first solid electrolyte layer 4 and the third substrate layer 3. The first through-hole conductor 63 is disposed in a first through-hole 69a that penetrates the first solid electrolyte layer 4, the spacer layer 5, and the second solid electrolyte layer 6 of the element body 102 in the up-down direction. The upper end of the first through-hole conductor 63 contacts the lower surface of the connector electrode 68c at the upper surface of the second solid electrolyte layer 6. The upper end of the first through-hole conductor 63 is entirely covered by the connector electrode 68c so as not to be exposed to the outside of the sensor element 101. The lower end of the first through-hole conductor 63 contacts the rear-end portion of the upper surface of the first lead wire 62 at the lower surface of the first solid electrolyte layer 4. A front-end portion of the first lead wire 62 is disposed in the reference gas chamber 49, and the front end of the first lead wire 62 contacts the first reference electrode 42a.

The first inside lead portion 61—more specifically, the first lead wire 62 and the first through-hole conductor 63—is a porous body through which the reference gas can pass internally. The connector electrode 68c is also a porous body through which the reference gas can pass internally. Therefore, the first inside lead portion 61 and the connector electrode 68c function as a reference gas flow path 47 that allows the reference gas to flow between the reference gas chamber 49 and the outside of the element body 102. The entirety of the first inside lead portion 61 constitutes a porous reference gas flow region 48 that forms part of the reference gas flow path 47. Thus, the reference gas can reach the reference gas chamber 49 from the outside of the element body 102 (the space 149 in FIG. 1) by passing, in this order, through the connector electrode 68c, the first through-hole conductor 63, and the first lead wire 62. Conversely, the reference gas in the reference gas chamber 49 can reach the space 149 by passing, in this order, through the first lead wire 62, the first through-hole conductor 63, and the connector electrode 68c.

In this way, because the reference gas flow path 47 exists, oxygen pumped into the reference gas chamber 49 by the reference-gas adjustment pump cell 31 can be vented to the outside of the sensor element 101 (the space 149 in FIG. 1) via the reference gas flow path 47. Therefore, it is possible to suppress an increase in oxygen concentration in the reference gas chamber 49. Accordingly, the inside of the reference gas chamber 49 is easily maintained at a predetermined oxygen concentration (here, the same oxygen concentration as that of atmospheric air). A change in oxygen concentration in the reference gas chamber 49 causes, for example, a change in voltage based on the first reference electrode 42a or the second reference electrode 42b, such as the voltage V2, which may affect the control of the sensor element 101 by the control device 90. As a result, the detection accuracy of the NOx concentration may deteriorate. Maintaining the inside of the reference gas chamber 49 at a predetermined oxygen concentration can suppress the deterioration of the detection accuracy of the NOx concentration.

The first lead wire 62 of the first inside lead portion 61 includes a first region 62a, a second region 62b, and a third region 62c. The first region 62a, the second region 62b, and the third region 62c are disposed in this order from the front-end side (the side of the first reference electrode 42a) toward the rear-end side (the side of the first through-hole conductor 63 and the connector electrode 68c). Each of the first to third regions 62a to 62c allow the reference gas to pass internally and constitute parts of the reference gas flow path 47. The second region 62b is configured as a low-porosity region having a low porosity. The first region 62a is configured as a high-porosity region having a higher porosity than the second region 62b, and connects the second region 62b and the first reference electrode 42a. That is, the porosity P1 of the first region 62a is higher than the porosity P2 of the second region 62b. The first region 62a is a region including the front-end portion of the first lead wire 62, a part of which is disposed in the reference gas chamber 49. The front end of the first region 62a contacts and is electrically conducted to the first reference electrode 42a. The third region 62c is a portion of the first lead wire 62 extending from the rear end of the second region 62b to the rear end of the first lead wire 62. The porosity P3 of the third region 62c is higher than the porosity P2 of the second region 62b. In this embodiment, the porosity P3 of the third region 62c is the same as the porosity P1 of the first region 62a. In this embodiment, the porosity P4 of the first through-hole conductor 63 and the porosity P5 of the connector electrode 68c are higher than the porosity P2 of the second region 62b and are the same as the porosity P1 of the first region 62a.

The second inside lead portion 65 is a conductor connected to the second reference electrode 42b and electrically conducts between the connector electrode 68g and the second reference electrode 42b. The second inside lead portion 65 includes a second lead wire 66 and a second through-hole conductor 67. The second lead wire 66 is formed on the upper surface of the second substrate layer 2 and is disposed so as to extend in the front-rear direction between the second substrate layer 2 and the third substrate layer 3. The second through-hole conductor 67 is disposed in a second through-hole 69b that penetrates the first substrate layer 1 and the second substrate layer 2 of the element body 102 in the up-down direction. The lower end of the second through-hole conductor 67 contacts the upper surface of the connector electrode 68g at the lower surface of the first substrate layer 1. The lower end of the second through-hole conductor 67 is entirely covered by the connector electrode 68g so as not to be exposed to the outside of the sensor element 101. The upper end of the second through-hole conductor 67 contacts the rear-end portion of the lower surface of the second lead wire 66 at the upper surface of the second substrate layer 2. A front-end portion of the second lead wire 66 is disposed in the reference gas chamber 49, and the front end of the second lead wire 66 contacts the second reference electrode 42b.

Unlike the first inside lead portion 61 and the connector electrode 68c, the second inside lead portion 65 and the connector electrode 68g are dense bodies rather than porous bodies, and do not allow the reference gas to flow therein. Therefore, the second inside lead portion 65 and the connector electrode 68g are not included in the reference gas flow path 47. Note that at least one of the second lead wire 66, the second through-hole conductor 67, and the connector electrode 68g may be a dense body, and the remaining ones may be porous bodies. Even in that case, since there is no path through which the reference gas can move between the outside of the sensor element 101 and the reference gas chamber 49 via these components, the second inside lead portion 65 and the connector electrode 68g are not included in the reference gas flow path 47.

Note that the first inside lead portion 61 and the second inside lead portion 65 are each covered with an insulating layer (not shown), and electrical insulation from the surrounding solid electrolyte layers is maintained.

Here, the sensor element 101 has a limiting current A of 1.5 μA or more when oxygen is pumped out from around the first reference electrode 42a to around the inner pump electrode 22 (an example of the first pump electrode). The method for measuring the limiting current A is as follows. First, the sensor element 101 is placed in a model gas having nitrogen as the base gas and 20% oxygen, and the heater 72 is energized to heat the sensor element 101 to a predetermined target temperature (for example, 800° C.). For example, the control unit 91 may perform the above-described heater control processing to control the heater 72 so that the first solid electrolyte layer 4 reaches the target temperature. The resistance value of the reference-gas adjustment pump cell 31 used for the heater control processing may be measured based on the values of a weak pump current Ip3 and the voltage V3 at that time by the control unit 91 controlling the power source circuit 36 so as to apply a relatively small voltage Vp3, as described above. After the temperature of the sensor element 101 stabilizes, the power source circuit 36 applies a voltage Vp3 between the inner pump electrode 22 and the first reference electrode 42a of the reference-gas adjustment pump cell 31 so that oxygen is pumped out from around the first reference electrode 42a to around the inner pump electrode 22, and the pump current Ip3 flowing between the two electrodes 22 and 42a is measured. The voltage Vp3 is a DC voltage. Thereafter, as the voltage Vp3 is gradually increased, the pump current Ip3 also gradually increases; however, when the voltage Vp3 is further increased, the pump current Ip3 ceases to increase and reaches an upper limit—that is, the pump current Ip3 becomes a limiting current. The value of the pump current Ip3 at this time is taken as the limiting current A. The limiting current A has a positive correlation with the reciprocal of the diffusion resistance of the reference gas flow path 47: the larger the limiting current A, the smaller the diffusion resistance of the reference gas flow path 47. That is, the limiting current A is a value representing structural characteristics of the reference gas flow path 47 of the sensor element 101. Since it is difficult to directly measure the diffusion resistance of the reference gas flow path 47 in an actual sensor element 101, the limiting current A is used as a value correlated with the diffusion resistance. By making this limiting current A 1.5 μA or more, the diffusion resistance between the outside of the element body 102 and the reference gas chamber 49 is not excessively large, and it is possible to suppress an increase in the oxygen concentration around the first reference electrode 42a (i.e., the oxygen concentration in the reference gas chamber 49) due to the oxygen pumping performed by the reference-gas adjustment pump cell 31. The limiting current A is preferably 2.0 μA or more. The limiting current A may be 4.0 μA or less, or 3.0 μA or less.

Adjustment of the limiting current A can be performed, for example, by adjusting at least one of the width, thickness, length, and porosities P1-P3 of the first lead wire 62; by adjusting at least one of the diameter, length, and porosity P4 of the first through-hole conductor 63; and/or by adjusting at least one of the thickness and porosity P5 of the connector electrode 68c.

The first and second lead wires 62 and 66, and the first and second through-hole conductors 63 and 67 each have a noble metal as a main component. “Main component” means a component that occupies a volumetric ratio of 50 vol % or more, or the component having the highest volumetric ratio among all components. The first and second lead wires 62 and 66, and the first and second through-hole conductors 63 and 67 may each be composed of a noble metal and unavoidable impurities. In this embodiment, Pt is used as the noble metal contained in each of the first and second lead wires 62 and 66 and the first and second through-hole conductors 63 and 67. The width of the first lead wire 62 may be, for example, 0.20 mm or more, or 0.25 mm or more. The width of the first lead wire 62 may be 0.60 mm or less, or 0.40 mm or less. The length of the first lead wire 62 may be 35 mm or more. The length of the first lead wire 62 may be 50 mm or less, or 45 mm or less. The thickness of the first lead wire 62 may be 10 μm or more. The thickness of the first lead wire 62 may be 30 μm or less, or 15 μm or less. The same applies to the width, length, and thickness of the second inside lead portion 65.

The connector electrodes 68a to 68h each have a noble metal as a main component. The connector electrodes 68a to 68h may each be composed of a noble metal and unavoidable impurities. In this embodiment, Pt is used as the noble metal contained in each of the connector electrodes 68a to 68h.

The porosities P1 to P3 described above for the first lead wire 62, the porosity P4 of the first through-hole conductor 63, and the porosity P5 of the connector electrode 68c may each be 1% or more. Each of the porosities P1, P3, P4, and P5 may be 5% or more, greater than 5%, or 10% or more. Each of the porosities P1, P3, P4, and P5 may be 25% or less, or 20% or less. The porosity P2 may be 10% or less, less than 10%, 5% or less, or less than 5%. As described above, in this embodiment the porosities P1, P3, P4, and P5 are set to the same value; however, some of these may be set to values different from the others. Further, at least one of the porosities P3 to P5 may be the same as or lower than the porosity P2. The second inside lead portion 65 and the connector electrode 68g are dense bodies as described above, and, for example, their porosities may be less than 1%.

The above-mentioned porosities such as porosities P1 to P5 are assumed to be values derived in the following way by using an image (SEM image) obtained through observation with a scanning electron microscope (SEM). First, the measurement target is cut so that the cross section of the measurement target is an observation surface, and the cut section is subjected to resin embedding and polishing to obtain a specimen for observation. Subsequently, a SEM photograph (secondary electron image at an acceleration voltage of 15 kV and a magnification of 1000) of the observation surface of the specimen for observation is taken to obtain an SEM image of the measurement target. Then, the obtained image is subjected to image analysis to determine a threshold value from the luminance distribution of luminance data of pixels in the image by using a discriminant analysis method (Otsu's binarization). Thereafter, each pixel in the image is binarized into a substance portion and a pore portion on the basis of the determined threshold value, and the area of the substance portion and the area of the pore portion are calculated. Then, the proportion of the area of the pore portion in the total area (the sum of the area of the substance portion and the area of the pore portion) is derived as a porosity (expressed in %).

As described above, the first lead wire 62 of the first inside lead portion 61 has the second region 62b as the low-porosity region. In this way, the presence of the second region 62b having the low porosity in part of the reference gas flow path 47, it is possible to suppress some gas outside the sensor element 101 from passing through the reference gas flow path 47 and reaching the reference gas chamber 49. As a result, it is possible to suppress a decrease in accuracy of NOx concentration detection by suppressing changes in the oxygen concentration in the reference gas chamber 49. As an example of some gas outside the sensor element 101, a volatile organic gas generated from the rubber stopper 157 when the sensor element 101 is exposed to a high-temperature environment can be cited. If such a gas reaches the reference gas chamber 49, the oxygen concentration in the reference gas chamber 49 decreases; however, the presence of the low-porosity second region 62b in the reference gas flow path 47 can suppress such gas from reaching the reference gas flow region 48.

In addition, the first lead wire 62 of the first inside lead portion 61 has the first region 62a as a high-porosity region between the low-porosity second region 62b and the first reference electrode 42a. Thus, the first region 62a functions as a buffer that suppresses rapid changes in the oxygen concentration in the reference gas chamber 49. Specifically, when the oxygen concentration in the reference gas chamber 49 rapidly decreases, oxygen can be supplied from the pores (space) in the first region 62a to the reference gas chamber 49; when the oxygen concentration in the reference gas chamber 49 rapidly increases, the pores (space) in the first region 62a can receive oxygen from the reference gas chamber 49. Therefore, the oxygen concentration in the reference gas chamber 49 can be stabilized.

It is preferable that the internal void volume Vh of the first region 62a be 0.5×10−3 mm³ or more. The void volume Vh can be calculated as a value obtained by dividing by 100 the product of the volume of the outer shape (for example, the product of width, length, and thickness) of the first region 62a and the porosity P1 [%]. With the void volume Vh being 0.5×10⁻³ mm³ or more, the first region 62a can more reliably exhibit the above-mentioned function as a buffer. The void volume Vh is preferably 1.0×10⁻³ mm³ or more, and more preferably 3.0×10⁻³ mm³ or more. The larger the void volume Vh, the higher the effect by which the first region 62a stabilizes the oxygen concentration in the reference gas chamber 49 becomes. The void volume Vh may be 10.0×10⁻³ mm³ or less, or 7.5×10−3 mm³ or less.

Next, the role played by the reference-gas adjustment pump cell 31 will be explained in detail. As described above, in the sensor element 101 the measurement pump cell 41 pumps oxygen into the reference gas chamber 49, and the flow rate of pumped oxygen varies according to the pump current Ip2, i.e., according to the NOx concentration in the measurement gas. By pumping oxygen with not only the measurement pump cell 41 but also the reference-gas adjustment pump cell 31, the total flow rate of oxygen pumped into the reference gas chamber 49 can be raised, thereby suppressing fluctuations in the oxygen concentration in the reference gas chamber 49 resulting from variations in the pump current Ip2. Note that when the pump current Ip2 is large, the oxygen concentration inside the reference gas chamber 49 tends to increase; however, because the limiting current A described above is 1.5 μA or more, it is possible to suppress oxygen from becoming excessive inside the reference gas chamber 49 and to suppress an increase in the oxygen concentration in the reference gas chamber 49. Conversely, when the pump current Ip2 is small, gas may flow from the outside of the sensor element 101 (the space 149) into the reference gas chamber 49 via the reference gas flow path 47. Normally, since the space 149 is in the same atmosphere as the reference gas, this is not a problem even if it occurs. However, for example, when some gas outside the sensor element 101, such as a volatile organic gas from the rubber stopper 157 described above, is present in the space 149, such a gas may enter the inside of the sensor element 101 via the reference gas flow path 47. In such a case, the presence of the above-described low-porosity second region 62b in the reference gas flow path 47 can prevent such gas from reaching the reference gas chamber 49 or reduce the flow rate of such gas reaching the reference gas chamber 49. This can suppress a decrease in the oxygen concentration in the reference gas chamber 49. Furthermore, the first region 62a functions as the above-described buffer to suppress rapid changes in the oxygen concentration in the reference gas chamber 49, thereby stabilizing the oxygen concentration in the reference gas chamber 49.

Next, an example method for manufacturing the gas sensor 100 will be described hereinafter. First, six unfired ceramic green sheets are prepared, each containing an oxygen-ion-conductive solid electrolyte such as zirconia as the ceramic component. A plurality of sheet holes used for positioning at the time of printing or stacking, a plurality of required through holes, and the like are formed in the green sheets in advance. A space that forms the measurement gas flow section is provided in advance in the green sheet for the spacer layer 5 by punching or the like. A space that forms the reference gas chamber 49 is provided in advance in the green sheet for the third substrate layer 3 by punching or the like. Then, the ceramic green sheets are subjected to a pattern printing process and a drying process in accordance with the first substrate layer 1, the second substrate layer 2, the third substrate layer 3, the first solid electrolyte layer 4, the spacer layer 5, and the second solid electrolyte layer 6 to form various patterns on the respective ceramic green sheets. Specifically, the patterns to be formed are patterns for, for example, the respective electrodes described above, lead wires to be connected to the respective electrodes, the connector electrodes, and the heater section 70. The pattern printing process is performed by applying pattern-forming paste, which is prepared according to the properties required for the respective objects to be formed, to the green sheets by using a known screen printing technique. The drying process is also performed using a known drying device. Upon completion of pattern printing and drying, an adhesive paste for stacking and bonding the green sheets corresponding to the respective layers is printed and dried. Then, a pressure bonding process is performed. Specifically, the respective green sheets with the adhesive paste formed thereon are positioned by aligning the sheet holes, stacked in a predetermined order, and then subjected to pressure bonding under predetermined temperature and pressure conditions to form a single layered body. The resulting layered body includes a plurality of sensor elements 101. The layered body is cut into the size of the sensor elements 101. Each of the cut pieces of the layered body is fired at a predetermined firing temperature to produce the sensor element 101. The patterns that will become the first through-hole conductor 63 and the second through-hole conductor 67 can be formed, for example, by a known through-hole printing either before or after lamination of the green sheets. The porosity P1 of the first region 62a of the first lead wire 62 can be adjusted by adjusting, for example, the proportion of a pore-forming material in the paste used for pattern formation of the first region 62a. Similarly, the porosity P2 of the second region 62b can be adjusted by adjusting, for example, the proportion of a pore-forming material in the paste used for pattern formation of the second region 62b. The porosities P3 to P5 can also be adjusted in the same manner.

After the sensor element 101 is obtained in the way described above, the sensor assembly 140 (see FIG. 1) with the sensor element 101 built therein is manufactured. The components, such as the protective cover 130 and the rubber stopper 157, are mounted in the sensor assembly 140, and the lead wires 155 are drawn out to the outside from the outer cylinder 148. Then, the control device 90 and the sensor element 101 are connected to each other via the lead wires 155. Thus, the gas sensor 100 is obtained.

Here, the correspondence relationship between the elements according to the present embodiment and the elements according to the present invention will be clarified. The element body 102 according to the present embodiment corresponds to the element body according to the present invention; the reference gas chamber 49 corresponds to the reference gas chamber; the inner pump electrode 22 corresponds to the first pump electrode; the first reference electrode 42a corresponds to the first reference electrode; the reference-gas adjustment pump cell 31 corresponds to the first pump cell; the connector electrode 68c corresponds to the first connector electrode; the first inner lead portion 61 corresponds to the first inner lead portion; the reference gas flow path 47 corresponds to the reference gas flow path; and the reference gas flow region 48 corresponds to the reference gas flow region. In addition, the second region 62b corresponds to the low-porosity region; the first region 62a corresponds to the high-porosity region; the measurement electrode 44 corresponds to the second pump electrode; the second reference electrode 42b corresponds to the second reference electrode; the measurement pump cell 41 corresponds to the second pump cell; the connector electrode 68g corresponds to the second connector electrode; and the second inner lead portion 65 corresponds to the second inner lead portion. Further, the protective cover 130 and the sensor assembly 140 correspond to the case, and the rubber stopper 157 corresponds to the sealing member.

In the gas sensor 100 according to this embodiment described in detail above, the limiting current A when oxygen is pumped from around the first reference electrode 42a to around the inner pump electrode 22 is 1.5 μA or more. Accordingly, because the diffusion resistance between the outside of the element body 102 and the reference gas chamber 49 is not excessive, so that an increase in the oxygen concentration around the first reference electrode 42a due to the pumping of oxygen by the reference-gas adjustment pump cell 31 can be suppressed. Moreover, when the limiting current A is 2.0 μA or more, the increase in the oxygen concentration around the first reference electrode 42a due to the pumping of oxygen by the reference-gas adjustment pump cell 31 can be further suppressed.

Further, the first inner lead portion 61 includes, as the part of the reference gas flow path 47, the second region 62b having the low porosity. As a result, it is possible to suppress some gas outside the sensor element 101 from passing through the reference gas flow path 47 and reaching the reference gas chamber 49. Consequently, the decrease in the detection accuracy of NOx concentration can be suppressed. In addition, the first inner lead portion 61 includes the first region 62a, having the higher porosity than the second region 62b, which connects the second region 62b to the first reference electrode 42a. Therefore, the first region 62a functions as the buffer that suppresses abrupt changes in the oxygen concentration in the reference gas chamber 49, and thus the oxygen concentration in the reference gas chamber 49 can be stabilized.

Furthermore, by setting the void volume Vh inside the first region 62a to 0.5×10⁻³ mm³ or more, the first region 62a can more reliably exhibit the buffer function described above.

It should be noted that the present invention is not limited to the present embodiment described above in any way, and it goes without saying that the present invention can be implemented in various aspects as long as they fall within the technical scope of the present invention.

For example, in the embodiment described above, the first inner lead portion 61 constituted a part of the reference gas flow path 47; however, it suffices that the first inner lead portion 61 constitute at least a part of the reference gas flow path 47. For instance, in the embodiment described above, the reference gas flow path 47 was constituted by the first inner lead portion 61 and the connector electrode 68c, but the reference gas flow path 47 may be constituted by only the first inner lead portion 61. For example, the connector electrode 68c may be a non-porous body and cover only a part of the upper end of the first through-hole conductor 63, with a part of the upper end of the first through-hole conductor 63 being exposed to the outside (space 149) of the sensor element 101. In this case, the connector electrode 68c is not included in the reference gas flow path 47, and only the first inner lead portion 61 (the first lead wire 62 and the first through-hole conductor 63) constitutes the reference gas flow path 47.

In the embodiment described above, the first through-hole conductor 63 was a porous body, but the present invention is not limited thereto. For example, the first through-hole conductor 63 may have a through-hole for allowing the reference gas to flow, and this through-hole may allow the reference gas to flow between the outside of the sensor element 101 and the first lead wire 62.

In the embodiment described above, the first inner lead portion 61 included the first lead wire 62 and the first through-hole conductor 63, but the present invention is not limited thereto. For example, the first inner lead portion 61 may not include the first through-hole conductor 63, and the first lead wire 62 may extend to the outer surface of the element body 102 (for example, the right surface or the left surface). In this case, it suffices to dispose, on the outer surface of the element body 102, an outer lead portion that electrically connects the first inner lead portion 61 (the first lead wire 62) and the connector electrode 68c.

In the embodiment described above, the first lead wire 62 of the first inner lead portion 61 included the first region 62a and the second region 62b, but the present invention is not limited thereto. For example, the first lead wire 62 may not include the second region 62b, and the entire first lead wire 62 may have the same porosity. Even in this case, if the limiting current A is 1.5 μA or more, an increase in the oxygen concentration around the first reference electrode 42a due to the pumping of oxygen by the reference-gas adjustment pump cell 31 can be suppressed. Alternatively, the first lead wire 62 may not include the first region 62a, and the second region 62b may be disposed up to inside the reference gas chamber 49 so as to be in contact with the first reference electrode 42a. Even in this case, if the limiting current A is 1.5 μA or more, the increase in the oxygen concentration around the first reference electrode 42a due to the pumping of oxygen by the reference-gas adjustment pump cell 31 can be suppressed. Moreover, by the presence, in a part of the reference gas flow path 47, of the second region 62b having a low porosity (for example, lower porosity than the third region 62c), it is possible to suppress some gas outside the sensor element 101 from passing through the reference gas flow path 47 and reaching the reference gas chamber 49.

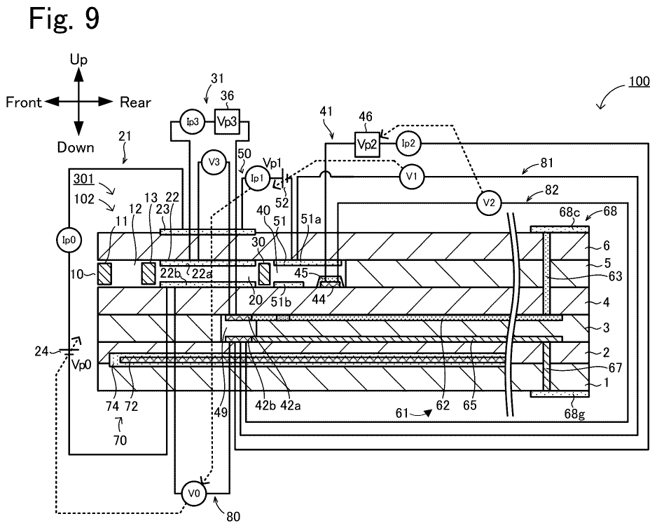

In the embodiment described above, the second inner lead portion 65 and the connector electrode 68g were dense bodies and are not included in the reference gas flow path 47; however, the present invention is not limited thereto, and they may be included in the reference gas flow path 47. For example, in the sensor element 201 according to a modification shown in FIG. 8, the second inner lead portion 265 (the second lead wire 266 and the second through-hole conductor 267) and the connector electrode 268g are configured as porous bodies and allow the reference gas to flow between the reference gas chamber 49 and the outside of the sensor element 201. Therefore, the second inner lead portion 265 and the connector electrode 268g constitute part of the reference gas flow path 47. In addition, the entirety of the second inner lead portion 265 constitutes the porous reference gas flow region 48 that forms a part of the reference gas flow path 47. In this sensor element 201, the reference gas flow path 47 has two flow paths: a flow path via the first inner lead portion 61 and the connector electrode 68c, and a flow path via the second inner lead portion 265 and the connector electrode 268g. In this embodiment, the limiting current A has a positive correlation with the reciprocal of the composite value of the diffusion resistances of these two flow paths. In this case as well, if the limiting current A is 1.5 μA or more, an increase in the oxygen concentration around the first reference electrode 42a due to the pumping of oxygen by the reference-gas adjustment pump cell 31 can be suppressed. In FIG. 8, the second lead wire 266 has no division into regions such as the first to third regions 62a to 62c of the first lead wire 62; the entire second lead wire 266 has the same porosity. However, the second lead wire 266 may also have first to third regions similar to the first lead wire 62.

In the sensor element 201 of FIG. 8, at least one of the first lead wire 62, the first through-hole conductor 63, and the connector electrode 68c may be a dense body so that these do not constitute the reference gas flow path 47. In this case, the measurement pump cell 41 corresponds to the first pump cell; the measurement electrode 44 corresponds to the first pump electrode; the second reference electrode 42b corresponds to the first reference electrode; and the second inner lead portion 265 corresponds to the first inner lead portion.

In the embodiment described above, both the first reference electrode 42a and the second reference electrode 42b were disposed in the reference gas chamber 49, but the present invention is not limited thereto. For example, the second reference electrode 42b may not be disposed in the reference gas chamber 49, and the first reference electrode 42a may also serve the role of the second reference electrode 42b. In this embodiment, the sensor element 101 does not include the second pump cell, the second connector electrode, or the second inner lead portion.

In the embodiment described above, in addition to the reference-gas adjustment pump cell 31, the measurement pump cell 41 performed oxygen intake toward the second reference electrode 42b; however, the present invention is not limited thereto. For example, the measurement pump cell 41 may be configured by the outer pump electrode 23, the measurement electrode 44, and, therebetween, the second solid electrolyte layer 6, the spacer layer 5, and the first solid electrolyte layer 4. In this case, the measurement pump cell 41 pumps oxygen from the measurement electrode 44 to the outer pump electrode 23. In this embodiment, the sensor element 101 does not include the second pump cell.

In the embodiment described above, the reference-gas adjustment pump cell 31 has been described as an example of the first pump cell, and the measurement pump cell 41 has been described as an example of the second pump cell. Alternatively, the first and second pump cells may be other pump cells for different uses that are configured to pump oxygen into the reference gas chamber 49. In the embodiment described above, furthermore, the layers 4 to 6 are present between the inner pump electrode 22 and the first reference electrode 42a included in the reference-gas adjustment pump cell 31. Alternatively, it is sufficient that the first pump cell includes one or more solid electrolyte layers between the first pump electrode and the first reference electrode. Likewise, it is sufficient that the second pump cell includes one or more solid electrolyte layers between the second pump electrode and the second reference electrode.

In the embodiment described above, the reference gas is atmospheric air. However, any other gas serving as a reference to detect the concentration of a specific gas in the measurement gas may be used. For example, the reference gas chamber 49 or the space 149 may be filled with a gas that is adjusted in advance to have a predetermined oxygen concentration (greater than the oxygen concentration in the measurement gas) as a reference gas.

In the embodiment described above, the sensor element 101 is configured to detect the NOx concentration in the measurement gas. Alternatively, any other device configured to detect the concentration of a specific gas in the measurement gas may be used. For example, any other oxide concentration other than NOx concentration may be detected as a concentration of a specific gas. When the specific gas is an oxide, as in the embodiment described above, the specific gas itself is reduced in the third internal cavity 60 to produce oxygen. Thus, the measurement pump cell 41 may obtain a detected value (i.e., the pump current Ip2) corresponding to the oxygen to detect the concentration of the specific gas. Alternatively, the specific gas may be a non-oxide such as ammonia. When the specific gas is a non-oxide, the specific gas is converted to an oxide (e.g., converted to NO in the case of ammonia) such that the converted gas is reduced in the third internal cavity 60 to produce oxygen. The measurement pump cell 41 can obtain a detected value (e.g., the pump current Ip2) corresponding to the oxygen to detect the concentration of the specific gas. For example, the inner pump electrode 22 in the first internal cavity 20 functions as a catalyst to convert ammonia to NO in the first internal cavity 20.

In the embodiment described above, the element body of the sensor element 101 is a layered body having a plurality of solid electrolyte layers (the layers 1 to 6), although this is not intended to be limiting. It is sufficient that the element body of the sensor element 101 includes at least one oxygen-ion-conductive solid electrolyte layer. For example, the first substrate layer 1 is not a constituent element of any cell and does not need to allow oxygen ions to pass. Thus, the first substrate layer 1 may be a layer composed of a material other than that of a solid electrolyte layer (e.g., a layer composed of alumina).

In the embodiment described above, the pump current Ip1 is used to control the voltage V0 of the main-pump-control oxygen-partial-pressure detection sensor cell 80, although this is not intended to be limiting. For example, feedback control of the pump voltage Vp0 may be performed on the basis of the pump current Ip1 so that the pump current Ip1 becomes the target value Ip1*. That is, the control of the voltage V0 based on the pump current Ip1 may be omitted, and the pump voltage Vp0 may be directly controlled (and therefore the pump current Ip0 may be controlled) on the basis of the pump current Ip1.

In the embodiment described above, the sensor element 101 of the gas sensor 100 includes the first internal cavity 20, the second internal cavity 40, and the third internal cavity 60, although this is not intended to be limiting. For example, as in a sensor element 301 illustrated in FIG. 9, the third internal cavity 60 may not be included. In the sensor element 301 according to a modification shown in FIG. 9, the gas inlet 10, the first diffusion rate-limiting section 11, the buffer space 12, the second diffusion rate-limiting section 13, the first internal cavity 20, the third diffusion rate-limiting section 30, and the second internal cavity 40 are formed adjacent to one another in such a manner as to communicate in the stated order between the lower surface of the second solid electrolyte layer 6 and the upper surface of the first solid electrolyte layer 4. The measurement electrode 44 is disposed on the upper surface of the first solid electrolyte layer 4 in the second internal cavity 40. The measurement electrode 44 is covered with a fourth diffusion rate-limiting section 45. The fourth diffusion rate-limiting section 45 is a film formed of a porous ceramic body composed of alumina (Al₂O₃) or the like. Like the fourth diffusion rate-limiting section 55 according to the embodiment described above, the fourth diffusion rate-limiting section 45 serves to limit the amount of NOx flowing into the measurement electrode 44. The fourth diffusion rate-limiting section 45 also functions as a protective film of the measurement electrode 44. The ceiling electrode portion 51a of the auxiliary pump electrode 51 is formed to extend up to the position immediately above the measurement electrode 44. The sensor element 301 having the configuration described above can also detect the NOx concentration on the basis of the pump current Ip2 in a way similar to that in the embodiment described above. In the sensor element 301 illustrated in FIG. 9, a surrounding portion of the measurement electrode 44 (the inside of the fourth diffusion rate-limiting section 45) functions as a measurement chamber. That is, the surrounding portion of the measurement electrode 44 implements functions similar to those of the third internal cavity 60.

In the embodiment described above, the gas sensor 100 may not include the control device 90. For example, the gas sensor 100 may include, instead of the control device 90, an external-connection connector attached to the lead wires 155 to connect the control device 90 and the lead wires 155 to each other.

### Examples

Hereinafter, examples in which a gas sensor was specifically manufactured will be described as Examples. Experimental Examples 1 to 3 and 5 to 12 correspond to examples of the present invention, and Experimental Example 4 corresponds to a comparative example. It should be noted that the present invention is not limited to the following Examples.

### Experimental Examples 1 to 4

A sensor element 101 of the gas sensor 100 shown in FIGS. 2, 4, and 5 to 7 was manufactured by the manufacturing method described above, and this was taken as Experimental Example 1. However, in Experimental Example 1, the first lead wire 62 did not include the second region 62b, and the entire first lead wire 62 had the same porosity. In manufacturing the sensor element 101, the ceramic green sheets were formed by tape casting of a mixture of zirconia particles to which 4 mol % of yttria as a stabilizer had been added, an organic binder, a dispersant, a plasticizer, and an organic solvent. The first reference electrode 42a was a porous cermet electrode of Pt and zirconia. The pattern for the first reference electrode 42a was formed using a paste prepared by mixing Pt powder, zirconia powder, a binder, and a pore-forming material. The materials of the first inner lead portion 61 and the connector electrode 68c were Pt. These patterns were formed using a platinum paste kneaded from platinum particles, a solvent, and a pore-forming material. The porosity of the first lead wire 62, the porosity P4 of the first through-hole conductor 63, and the porosity P5 of the connector electrode 68c were each set to 15%. For the sensor element 101 of Experimental Example 1, the limiting current A measured by the method described above was 2.0 μA. Further sensor elements 101 were manufactured as Experimental Examples 2-4 in the same manner as Experimental Example 1 except that the width, length, and thickness of the first lead wire 62 were appropriately changed to vary the limiting current A. The limiting current A was 4.0 μA in Experimental Example 2, 1.5 μA in Experimental Example 3, and 1.0 μA in Experimental Example 4.

### Experimental Examples 5 to 10

Except that the first lead wire 62 included the second region 62b as shown in FIGS. 2, 4, and 5 to 7 (and therefore included the first region 62a and the third region 62c as well), the sensor element 101 was manufactured in the same manner as in Experimental Examples 1 to 4, and this was taken as Experimental Example 5. In Experimental Example 5, the porosity P2 of the second region 62b was set to 5%, and the porosities P1, P3, P4, and P5 of the first region 62a, the third region 62c, the first through-hole conductor 63, and the connector electrode 68c were each set to 15%. The void volume Vh of the first region 62a of Experimental Example 5 was set to 0.5×10⁻³ mm³. The limiting current A of Experimental Example 5 was 2.0 μA. In addition, sensor elements 101 were manufactured in the same manner as in Experimental Example 5 as Experimental Examples 6 to 9, except that the width, length, and thickness of the first region 62a were appropriately changed to vary the void volume Vh, and further, the width, length, and thickness of the portions of the first lead wire 62 other than the first region 62a were also appropriately changed so that the limiting current A did not change from that of Experimental Example 5. Specifically, the void volume Vh was set to 1.0×10⁻³ mm³ in Experimental Example 6; specifically, the void volume Vh was set to 3.6×10⁻³ mm³ in Experimental Example 7; specifically, the void volume Vh was set to 7.2×10⁻³ mm³ in Experimental Example 8; and specifically, the void volume Vh was set to 10.0×10⁻³ mm³ in Experimental Example 9. The limiting current A was 2.0 μA in each of Experimental Examples 6 to 9. Further, a sensor element 101 was manufactured in the same manner as in Experimental Example 7 as Experimental Example 10, except that, while the void volume Vh was set to the same value as in Experimental Example 7, the width, length, and thickness of the portions of the first lead wire 62 other than the first region 62a were appropriately changed to vary the limiting current A from that in Experimental Example 7. That is, the void volume Vh of Experimental Example 10 was 3.6×10⁻³ mm³. The limiting current A of Experimental Example 10 was 1.5 ρA.

### Experimental Example 11

A sensor element 101 identical to that of Experimental Example 2 was manufactured as Experimental Example 11.

### Experimental Example 12

Except that the porosity P1 of the first region 62a was set to the same value as the porosity P2 of the second region 62b—i.e., the first lead wire 62 substantially did not include the first region 62a, and the second region 62b was also disposed within the reference gas chamber 49 so as to contact the first reference electrode 42a—the sensor element 101 was manufactured in the same manner as in Experimental Examples 5 to 9, and this was taken as Experimental Example 12. Therefore, in Experimental Example 12, since the first lead wire 62 does not have a region (first region 62a) between the first reference electrode 42a and the second region 62b, the void volume Vh is 0.0×10⁻³ mm³. The limiting current A in Experimental Example 12 was 1.8 μA.

### [Evaluation Test 1]

For the sensor elements 101 of Experimental Examples 1 to 4, 7, and 10, the oxygen concentration in the reference gas chamber 49 when the reference-gas adjustment pump cell 31 pumped oxygen into the reference gas chamber 49 was evaluated (Evaluation Test 1). Specifically, first, the sensor element 101 of Experimental Example 1 was incorporated into the gas sensor 100 shown in FIGS. 1-3, and the gas sensor 100 was attached to a pipe 190. A model gas was flowed through the pipe 190 as the measurement gas, the base gas being nitrogen and the NOx concentration being 500 ppm. In this state, the control of the respective pump cells 21, 31, 41, and 50 described above, the heater control processing, acquisition of the voltages V0, V1, and V2 from the respective sensor cells 80 to 82 described above, and measurement of the resistance value of the reference-gas adjustment pump cell 31 by the control device 90, were continued. The control device 90 controlled the reference-gas adjustment pump cell 31 such that oxygen was pumped from the first internal cavity 20 into the reference gas chamber 49 and the value of the pump current Ip3 became 5 μA. The state in which the model gas was flowed through the pipe 190 and the state in which the control device 90 controlled the sensor element 101 were maintained for 30 minutes, and the pump current Ip2 during that period was measured. Taking the pump current Ip2 at the start of measurement as 100%, the degree to which the pump current Ip2 decreased from the start of measurement was investigated. Specifically, when the pump current Ip2 remained within a prescribed range even after 30 minutes from the start of measurement, it was determined that the increase in the oxygen concentration in the reference gas chamber 49 was sufficiently suppressed (“A”). When the pump current Ip2 remained within the prescribed range until 20 minutes from the start of measurement but fell below the lower limit of the prescribed range before 30 minutes elapsed, it was determined that the increase in the oxygen concentration in the reference gas chamber 49 was suppressed to a certain extent (“B”). When the pump current Ip2 fell below the lower limit of the prescribed range before 20 minutes elapsed from the start of measurement, it was determined that suppression of the increase in the oxygen concentration in the reference gas chamber 49 was insufficient (“C”). Evaluation Test 1 was carried out for Experimental Examples 2-4, 7, and 10 in the same manner. Here, in Evaluation Test 1 the NOx concentration of the measurement gas is constant; theoretically, the pump current Ip2 also remains constantly at a fixed value. However, when the reference-gas adjustment pump cell 31 intakes oxygen, the oxygen concentration in the reference gas chamber 49 increases, and voltages referenced to the first reference electrode 42a or the second reference electrode 42b, such as the voltage V2, change. For example, when the oxygen concentration in the reference gas chamber 49 increases, the absolute value of the voltage V2 becomes larger even if the oxygen concentration in the third internal cavity 60 is the same. Therefore, even if the voltage Vp2 of the power source circuit 46 is feedback-controlled so that the voltage V2 becomes the target value V2*, the oxygen concentration in the third internal cavity 60 is not lowered sufficiently; that is, pumping out of the oxygen generated in the third internal cavity 60 by reduction of NOx becomes insufficient, and the pump current Ip2 decreases. Accordingly, the more the pump current Ip2 decreases within 30 minutes in Evaluation Test 1, the more the oxygen concentration in the reference gas chamber 49 is considered to be increasing.

### [Evaluation Test 2]

For the sensor elements 101 of Experimental Examples 5 to 12, the effect of volatile organic gases outside the sensor element 101 on the measurement accuracy of NOx concentration was evaluated (Evaluation Test 2). Specifically, first, as in Evaluation Test 1 described above, the sensor element 101 of Experimental Example 5 was incorporated into the gas sensor 100, and the gas sensor 100 was attached to the pipe 190. The rubber stopper 157 of the gas sensor 100 was made of fluororubber. The gas sensor 100 was heated to raise its temperature such that the temperature of the rubber stopper 157 became 250° C. After the start of the temperature rise, a model gas having a constant NOx concentration, as in Evaluation Test 1, was flowed as the measurement gas through the pipe 190, and control by the control device 90 was continued. Then, the average value μ and the standard deviation σ of the pump current Ip2 during the period from the time 3 minutes after the start of control by the control device 90 until the time when the rubber stopper 157 reached 250° C. were determined, and the average value μ and the standard deviation σ were used as indices for evaluating the measurement accuracy of the NOx concentration. Specifically, when the pump current Ip2 at the end of the test, 90 minutes after the start of heating of the rubber stopper 157, fell within the range μ±2σ, it was determined that the decrease in the measurement accuracy of NOx concentration was sufficiently suppressed (“A”). When the pump current Ip2 at the end of the test did not fall within the range μ+2σ but fell within the range μ±4σ, it was determined that the decrease in measurement accuracy of NOx concentration was suppressed to a certain extent (“B”). When the pump current Ip2 at the end of the test did not fall within the range μ+4σ, it was determined that suppression of the decrease in measurement accuracy of NOx concentration was insufficient (“C”). It should be noted that even if the pump current Ip2 deviated from the above range at a time prior to the end of the test, such deviations were excluded from judgment because they might be attributable to foreign matter adhering to the gas sensor 100. Evaluation Test 2 was carried out for Experimental Examples 6 to 12 in the same manner. Here, in Evaluation Test 2, because heating of the rubber stopper 157 generates volatile organic gases, if these volatile organic gases reach the reference gas chamber 49, the oxygen concentration in the reference gas chamber 49 decreases. When the oxygen concentration in the reference gas chamber 49 decreases, voltages referenced to the first reference electrode 42a or the second reference electrode 42b, such as the voltage V2, change. For example, when the oxygen concentration in the reference gas chamber 49 decreases, the absolute value of the voltage V2 becomes smaller even if the oxygen concentration in the third internal cavity 60 is the same. Therefore, when the voltage Vp2 of the power source circuit 46 is feedback-controlled such that the voltage V2 becomes the target value V2*, pumping-out of oxygen from the third internal cavity 60 becomes excessive, and the pump current Ip2 increases. As a result, the measurement accuracy of NOx concentration decreases. Accordingly, the more the pump current Ip2 falls outside the above ranges in Evaluation Test 2, the more the volatile organic gases are considered to reach the reference gas chamber 49.

### [Evaluation Test 3]

For the sensor elements 101 of Experimental Examples 5 to 12, the stability of the oxygen concentration in the reference gas chamber was evaluated (Evaluation Test 3). Specifically, first, as in Evaluation Test 1 described above, the sensor element 101 of Experimental Example 5 was incorporated into the gas sensor 100, and the gas sensor 100 was attached to the pipe 190. Then, a model gas with an NOx concentration of 0 ppm was flowed through the pipe 190 as the measurement gas, and control by the control device 90 was continued as in Evaluation Test 1. Subsequently, the NOx concentration of the model gas was abruptly changed from 0 ppm to 500 ppm. The difference between the pump current Ip2 at 10 seconds after the abrupt change and the pump current Ip2 immediately before the abrupt change was taken as ΔIp2a. Similarly, the NOx concentration of the model gas was abruptly changed from 0 ppm to 1500 ppm, and the difference between the pump current Ip2 at 10 seconds after the abrupt change and the pump current Ip2 immediately before the abrupt change was taken as ΔIp2b. The linearity [%] of the change in the pump current Ip2 was calculated by Equation (1) below. When this linearity was 90% or higher, it was determined that the stability of the oxygen concentration in the reference gas chamber 49 was very high (“A”). When the linearity was 80% or higher and less than 90%, it was determined that the stability of the oxygen concentration in the reference gas chamber 49 was high (“B”). When the linearity was less than 808, it was determined that the stability of the oxygen concentration in the reference gas chamber 49 was low (“C”). Evaluation Test 3 was carried out for Experimental Examples 6-12 in the same manner. Here, when the NOx concentration in the measurement gas abruptly changes, the pump current Ip2 also abruptly changes; however, if the oxygen concentration in the reference gas chamber 49 is constant, the change in the pump current Ip2 due to the abrupt change is theoretically linear. Specifically, when plotting, on a graph with NOx concentration on the horizontal axis and pump current Ip2 on the vertical axis, the Ip2 value for an NOx concentration of 0 ppm, the Ip2 value for 500 ppm, and the Ip2 value for 1500 ppm, these are theoretically located on the same straight line; i.e., the linearity is 100%. However, because the measurement pump cell 41 pumps oxygen into the reference gas chamber 49, when the pump current Ip2 abruptly increases, the flow rate of oxygen pumped into the reference gas chamber 49 also abruptly increases. If this causes the oxygen concentration in the reference gas chamber 49 to increase rapidly, the pump current Ip2 becomes smaller than its theoretical value as explained in Evaluation Test 1. Moreover, the higher the NOx concentration abruptly becomes, the more easily the pump current Ip2 becomes much smaller than its theoretical value; thus, in an actual sensor element 101, the linearity is basically less than 100%. In particular, the more the oxygen concentration in the reference gas chamber 49 tends to abruptly change accompanying an abrupt change in the flow rate of oxygen pumped into the reference gas chamber 49, the lower the linearity becomes. Therefore, the higher (closer to 100%) the linearity is, the less likely abrupt changes in the oxygen concentration of the reference gas chamber occur; that is, the higher the stability of the oxygen concentration in the reference gas chamber.

$$Linearity = ( Δ Ip 2 b / 1500 ) / ( Δ Ip 2 a / 500 ) \times 100 \qquad ( 1 )$$

For each of Experimental Examples 1 to 4, 7, and 10, Table 1 shows the value of the limiting current A, the presence/absence of the low-porosity region (second region 62b), and the judgment result of Evaluation Test 1. For each of Experimental Examples 5-12, Table 2 shows the value of the limiting current A, the presence/absence of the low-porosity region (second region 62b), the value of the void volume Vh, and the judgment results of Evaluation Tests 2 and 3. It should be noted that in Experimental Example 11, since the second region 62b is absent, the first region 62a that connects the first reference electrode 42a and the second region 62b cannot be defined; therefore, the field for the void volume Vh is left blank (“-”).

TABLE 1

| | Limiting | Low-porosity | |
|---|---|---|---|
| | Current | Region (Second | Evaluation |
| | A [μA] | Region 62b) | Test 1 |
| Experimental Example 1 | 2.0 | Absence | A |
| Experimental Example 2 | 4.0 | Absence | A |
| Experimental Example 3 | 1.5 | Absence | B |
| Experimental Example 4 | 1.0 | Absence | C |
| Experimental Example 7 | 2.0 | Presence | A |
| Experimental Example 10 | 1.5 | Presence | B |

TABLE 2

| | Limiting | Low-porosity Region | Void Volume Vh | Evaluation | Evaluation |
|---|---|---|---|---|---|
| | Current A [μA] | (Second Region 62b) | [×10⁻³ mm³] | Test 2 | Test 3 |
| Experimental Example 5 | 2.0 | Presence | 0.5 | A | B |
| Experimental Example 6 | 2.0 | Presence | 1.0 | A | A |
| Experimental Example 7 | 2.0 | Presence | 3.6 | A | A |
| Experimental Example 8 | 2.0 | Presence | 7.2 | A | A |
| Experimental Example 9 | 2.0 | Presence | 10.0 | B | A |
| Experimental Example 10 | 1.5 | Presence | 3.6 | A | A |
| Experimental Example 11 | 4.0 | Absence | — | C | A |
| Experimental Example 12 | 1.8 | Presence | 0.0 | A | C |

As shown in Table 1, while Experimental Example 4, in which the limiting current A was 1.0 μA, had a judgment result of “C” in Evaluation Test 1, Experimental Examples 1 to 3, in which the limiting current A was 1.5 μA or more, had judgment results of “B” or “A” in Evaluation Test 1. From these results, it was confirmed that with a limiting current A of 1.5 μA or more, an increase in the oxygen concentration in the reference gas chamber 49 due to the pumping of oxygen by the reference-gas adjustment pump cell 31 can be suppressed. Further, from a comparison between Experimental Examples 1 and 2 and Experimental Example 3, it is considered preferable that the limiting current A be 2.0 μA or more. It was also confirmed-based on a comparison between Experimental Examples 1-3, which did not have the second region 62b, and Experimental Examples 7 and 10, which had the second region 62b—that regardless of the presence/absence of the second region 62b, if the value of the limiting current A is the same, the results of Evaluation Test 1 are the same. This is considered to be because, as described above, the limiting current A has a positive correlation with the reciprocal of the diffusion resistance of the reference gas flow path 47, and therefore the effect of the presence/absence of the second region 62b on the diffusion resistance of the reference gas flow path 47 is also reflected in the limiting current A.

As shown in Table 2, while Experimental Example 11, which does not have the second region 62b, had a judgment result of “C” in Evaluation Test 2, Experimental Examples 5-10 and 12, which have the second region 62b, had judgment results of “B” or “A” in Evaluation Test 2. From these results, it was confirmed that the presence, in a part of the reference gas flow path 47, of the low-porosity region serving as the second region 62b can suppress volatile organic gases outside the sensor element 101 from passing through the reference gas flow path 47 and reaching the reference gas chamber 49.

As shown in Table 2, while Experimental Example 12, which does not have the first region 62a, had a judgment result of “C” in Evaluation Test 3, Experimental Examples 5-10, which have the first region 62a, had judgment results of “B” or “A” in Evaluation Test 3. From these results, it was confirmed that the first region 62a, serving as a high-porosity region, functions as a buffer that suppresses abrupt changes in the oxygen concentration of the reference gas chamber 49 and can stabilize the oxygen concentration in the reference gas chamber 49. Furthermore, from the results of Experimental Examples 5 to 10, it was confirmed that the first region 62a exhibits its function as a buffer when the void volume Vh is at least 0.5×10⁻³ mm³. In addition, from a comparison between Experimental Example 5 and Experimental Examples 6 to 10, it is considered preferable that the void volume Vh be 1.0×10⁻³ mm³ or more.

What is claimed is:

1. A sensor element for detecting a concentration of a specific gas in a measurement gas, the sensor element comprising:
an element body having an oxygen-ion-conductive solid electrolyte layer and having formed therein a measurement gas flow path and a reference gas chamber, the measurement gas flow path being a path into and through which the measurement gas is introduced and flows, the reference gas chamber being a chamber in which a reference gas used as a reference to detect the concentration of the specific gas in the measurement gas is to be stored;
a first pump cell having a first pump electrode disposed at a portion of the element body that comes into contact with the measurement gas, and a first reference electrode disposed in the reference gas chamber, the first pump cell being configured to pump oxygen from around the first pump electrode to around the first reference electrode;
a first connector electrode disposed on an outer surface of the element body; and
a first inner lead portion disposed inside the element body, connected to the first reference electrode, and configured to electrically connect the first connector electrode and the first reference electrode;
wherein the first inner lead portion has a reference gas flow region that is porous and constitutes at least part of a reference gas flow path allowing the reference gas to flow between the reference gas chamber and an outside of the element body; and
wherein a limiting current A when oxygen is pumped out from around the first reference electrode to around the first pump electrode is 1.5 μA or more.

2. The sensor element according to claim 1,
wherein the limiting current A is 2.0 μA or more.

3. The sensor element according to claim 1,
wherein the first inner lead portion has a low-porosity region constituting part of the reference gas flow path, and a high-porosity region having a porosity higher than that of the low-porosity region and connecting the low-porosity region and the first reference electrode.

4. The sensor element according to claim 3,
wherein a void volume Vh inside the high-porosity region is 0.5×10⁻³ mm³ or more.

5. The sensor element according to claim 4,
wherein the void volume Vh is 1.0×10⁻³ mm³ or more.

6. The sensor element according to claim 1, further comprising:
a second pump cell having a second pump electrode disposed at a portion of the element body that comes into contact with the measurement gas, and a second reference electrode disposed in the reference gas chamber, the second pump cell being configured to pump oxygen from around the second pump electrode to around the second reference electrode;
a second connector electrode disposed on an outer surface of the element body; and
a second inner lead portion disposed inside the element body, connected to the second reference electrode, and configured to electrically connect the second connector electrode and the second reference electrode.

7. A gas sensor comprising the sensor element according to claim 1.

8. The sensor element according to claim 2,
wherein the first inner lead portion has a low-porosity region constituting part of the reference gas flow path, and a high-porosity region having a porosity higher than that of the low-porosity region and connecting the low-porosity region and the first reference electrode.

9. The sensor element according to claim 2, further comprising:
a second pump cell having a second pump electrode disposed at a portion of the element body that comes into contact with the measurement gas, and a second reference electrode disposed in the reference gas chamber, the second pump cell being configured to pump oxygen from around the second pump electrode to around the second reference electrode;
a second connector electrode disposed on an outer surface of the element body; and
a second inner lead portion disposed inside the element body, connected to the second reference electrode, and configured to electrically connect the second connector electrode and the second reference electrode.

10. The sensor element according to claim 3, further comprising:
a second pump cell having a second pump electrode disposed at a portion of the element body that comes into contact with the measurement gas, and a second reference electrode disposed in the reference gas chamber, the second pump cell being configured to pump oxygen from around the second pump electrode to around the second reference electrode;
a second connector electrode disposed on an outer surface of the element body; and
a second inner lead portion disposed inside the element body, connected to the second reference electrode, and configured to electrically connect the second connector electrode and the second reference electrode.

11. The sensor element according to claim 4, further comprising:
a second pump cell having a second pump electrode disposed at a portion of the element body that comes into contact with the measurement gas, and a second reference electrode disposed in the reference gas chamber, the second pump cell being configured to pump oxygen from around the second pump electrode to around the second reference electrode;
a second connector electrode disposed on an outer surface of the element body; and
a second inner lead portion disposed inside the element body, connected to the second reference electrode, and configured to electrically connect the second connector electrode and the second reference electrode.

12. The sensor element according to claim 5, further comprising:
a second pump cell having a second pump electrode disposed at a portion of the element body that comes into contact with the measurement gas, and a second reference electrode disposed in the reference gas chamber, the second pump cell being configured to pump oxygen from around the second pump electrode to around the second reference electrode;
a second connector electrode disposed on an outer surface of the element body; and
a second inner lead portion disposed inside the element body, connected to the second reference electrode, and configured to electrically connect the second connector electrode and the second reference electrode.

13. The sensor element according to claim 8, further comprising:
a second pump cell having a second pump electrode disposed at a portion of the element body that comes into contact with the measurement gas, and a second reference electrode disposed in the reference gas chamber, the second pump cell being configured to pump oxygen from around the second pump electrode to around the second reference electrode;
a second connector electrode disposed on an outer surface of the element body; and
a second inner lead portion disposed inside the element body, connected to the second reference electrode, and configured to electrically connect the second connector electrode and the second reference electrode.
